SEQUENCE LISTING (5 sequences)

SEQ ID NO: 1 - DNA, 67 bp, Artificial Sequence
  Description of Artificial Sequence Synthetic primer
gttaatatacctctatactttaacgtcaaggagaaaaaactataatggagctcgttttcgacactgg

SEQ ID NO: 2 - DNA, 68 bp, Artificial Sequence
  Description of Artificial Sequence Synthetic primer
gaaaaaaatgagaagttgttctgaacaaagtaaaaaaaagaagtatactccttaccattaagttgatc

SEQ ID NO: 3 - DNA, 18 bp, Artificial Sequence
  Description of Artificial Sequence Synthetic primer
ggccccacaaaccttcaa

SEQ ID NO: 4 - DNA, 20 bp, Artificial Sequence
  Description of Artificial Sequence Synthetic primer
atgtcaaggtcttctcgagg

SEQ ID NO: 5 - DNA, 19 bp, Artificial Sequence
  Description of Artificial Sequence Synthetic primer
aatattgttgatgcgctgg

(19) United States
(12) Patent Application Publication
(10) Pub. No.: US 2009/0047708 A1
(43) Pub. Date: Feb. 19, 2009

(54) HIGHLY PRODUCTIVE RECOMBINANT YEAST STRAINS WITH MODIFIED GALACTOSE-REGULATED TRANSCRIPTION

(76) Inventors: Mateja Novak Stagoj, Ljubljana (SI); Sasa Comino, Ljubljana (SI); Zarko Lenardic, Ljubljana (SI); Radovan Komel, Ljubljana (SI)

Correspondence Address: SMITH MOORE LEATHERWOOD LLP, P.O. BOX 21927, GREENSBORO, NC, 27420, US

(21) Appl. No.: 11/913,028

(22) PCT Filed: Apr. 14, 2006
(86) PCT No.: PCT/SI2006/000011

§ 371 Date: Jul. 30, 2008

(30) Foreign Application Priority Data
May 5, 2005 (SI) P-200500131

(51) Int. Cl. C12P 21/04; C12N 1/19

(52) U.S. Cl. 435/69.1; 435/254.2; 435/254.21

(57) ABSTRACT

A recombinant yeast cell with modified galactose-regulated transcription, a use of such yeast cell, and products made using such yeast cell are disclosed. In such a recombinant yeast cell, a chromosomal gene encoding an enzyme for transformation of a transcription inductor into an inactive product is replaced by a gene encoding a transcription factor for a galactose-inducible promoter. Examples of yeast cells include those from Saccharomycetaceae or Cryptococcaceae families or from the Saccharomyces genus. The GAL1 gene encodes the enzyme for the galactose metabolism, and the GAL4 gene encodes the transcription factor. The galactose-inducible promoter is regulated by the Gal4 transcription factor, and regulates the transcription of GAL1, GAL2, GAL7, GAL10 or MEL1. Recombinant yeast cells include hosts for plasmids applicable in heterologous gene expression from the galactose-inducible promoter, and can be used in the production of bioactive substances, and other products of biotechnology.

This application is a national stage application under 35 U.S.C. 371 of international application No. PCT/SI2006/000011 filed Apr. 14, 2006, and claiming priority to Slovenian Application No. P-200500131 filed May 5, 2005, the disclosures of which are expressly incorporated herein by reference.

### TECHNICAL FIELD OF THE INVENTION

The present invention is directed to highly productive recombinant yeast strains with modified galactose-regulated transcription, to a method for producing bioactive substances, and to their use. The invention relates to the field of biotechnology. The fundamental goal of biotechnology is the industrial utilization of biocultures (microorganisms, tissue and cell cultures), to obtain various biotechnical products, useful for food industry, and pharmaceutical industry. The yeast Saccharomyces cerevisiae is an important host for production of a variety of heterologous proteins that have been used for the production of therapeutic and diagnostic agents, and as vaccines. Saccharomyces cerevisiae is considered as a GRAS (Generally Regarded as Safe) organism. In addition to safe use, the yeast Saccharomyces cerevisiae is the most thoroughly investigated eukaryotic microorganism.

### SUMMARY OF THE INVENTION

The object of this invention is a microbiological method, wherein the microbiological material is subjected to manipulation. The salient feature of this invention is the replacement of the GAL1 gene with the GAL4 gene in the chromosome of the yeast Saccharomyces cerevisiae. The locus of the GAL1 gene encoding the galactokinase enzyme is, by means of homologous recombination, replaced with the gene for the Gal4 transcription activator. The designed gene replacement causes in the mutant S. cerevisiae strain a significantly improved synthesis of recombinant proteins, expressed under the control of the GAL1 promoter.

The overall procedure of GAL genes replacement in the genome, which is the object of this invention, yields highly productive recombinant S. cerevisiae strains that are applicable in the expression of industrially (pharmaceutically) interesting proteins. By the utilization of the recombinant strain, which is an object of this invention, the inputs are significantly diminished (galactose is a relatively expensive inductor), and simultaneously a high level production of the desired recombinant protein is achieved.

### STATE OF THE ART

The yeast S. cerevisiae represents an important tool for the production of recombinant proteins. Recombinant yeasts were used in the production of human interferon for the first time in 1981. One year later, recombinant vaccine for hepatitis B was produced, for the first time, with the aid of yeasts. Today, Insulin obtained from recombinant S. cerevisiae supplies half of the world's needs.

Gene expression is a complex, multi-stage process. Among the main factors, affecting the specific productivity, are promoter strength, plasmid copy number, and the presence of the 5′-untranslated leader sequences. Comprehensive research on gene regulation in yeasts considerably contributed to the understanding of gene expression mechanism in higher eukaryots. Best-investigated mechanisms in yeasts comprise, with certainty, the GAL genes regulatory mechanism. The major features of the GAL model were established by Douglas and Hawthorne in the seventies.

GAL1, GAL7, and GAL10 genes are clustered near the centromere of chromosome II, while GAL4, GAL80, and GAL3 regulatory genes are located on separate chromosomes. The GAL4 gene is located on chromosome XVI. The Gal4 protein plays a key role in the regulation of GAL genes, as it is required for the expression of GAL1, GAL2, GAL7, and GAL10 genes. Consequently, the mutation in GAL4 prevents the transcription of the genes. Tightly regulated GAL1, GAL7, and GAL10 genes encode proteins (galactokinase, transferase, and epimerase), crucial to galactose metabolism. An important enzyme in galactose metabolism is galactokinase (EC 2.7.1.6), catalyzing the conversion of the inductor galactose into galactose-1-phosphate. Galactose is transported into the yeast cell with a specific permease, encoded by the GAL2 gene. The expression of GAL genes is precisely regulated: in the presence of galactose the expression of these genes is highly enhanced, even up to 1000-fold, while glucose acts as a potent repressor (inhibitor) on the expression thereof. The growth in a glucose containing medium inhibits, even in the presence of galactose, the expression of GAL genes. This phenomenon is termed catabolite repression.

Promoter sequences of GAL1, GAL7, and GAL10 genes regulating high expression of the genes belong to the best-investigated S. cerevisiae. They are important for the design and construction of vectors. Known are two crucial advantages of the utilization of GAL promoters. In the first place, the possibility to grow cells on a nutrient medium where the activity of both promoters is practically repressed, which enables an appropriate cell density in the culture (biomass), and successive induction. Thus, the production of proteins, potentially toxic for the yeast cell, becomes feasible. Secondly, such promoters are among the strongest inducible promoter in yeast S. cerevisiae.

A positive regulator is the Gal4 transcription factor, which is bound to promoter sequences of these genes. In the presence of glucose, the negative regulator Gal80 represses the induction by means of binding to GAL4. The expression of Gal4 in the cell is minimized to only one to two molecules. For an optimal induction of GAL promoters, a sufficient Gal4 protein level in the cell is, however, indispensable. This is especially important for the production of heterologous proteins expressed from multi-copy plasmids.

The expression level of the target protein is to a large extent determined by inherent characteristics of the heterologous protein. The yield, however, may be considerably improved by taking into account various factors influencing gene expression, and product stability. In the scientific literature are disclosed several attempts of genetic engineering of the GAL system, with the aim to optimize fermentation processes.

Since the Gal4 transcription factor is present in very low concentrations in the cell, whereby the GAL genes induction is limited, researchers attempted to surmount this obstacle. The solution is not a constitutive over-expression of GAL4, as it results in the loss of regulation. To avoid this drawback, a controlled expression is indispensable. Laughon et al. described in the journal Molecular and Cellular Biology 4, 268-275 in 1984, transformed yeast strains that contained multi-copy plasmids, with the GAL4 structural gene fused to the GAL1 promoter. The scientific literature and patent specifications disclose examples for the integration of GAL4 with the GAL10 promoter in the yeast S. cerevisiae genome. U.S. Pat. No. 5,068,185 describes the integration of the GAL10-GAL4 expression cassette into the HIS3 chromosomal locus. A three-fold increase in production of the target protein was observed in the presence of galactose (U.S. Pat. No. 5,068,185), (Schultz et al., 1987).

The use of productive strains mutated in GAL1 gene was disclosed by Hovland et al. in 1989 in the journal Gene, 15, 57-64, and later by several others. They found that in mutant strains (gal1), incapable of metabolic transformation of the inductor, the target genes expression driven by galactose-regulated promoters was significantly enhanced. In gal1 strains, the galactose level in fermentation cultures remains unaltered, enabling and warranting a steady and constant induction of GAL promoters.

We solved the problem of the state of the art processes, which required high galactose levels in the culture medium for the production of bioactive substances, with gene replacement in a manner enabling an enhanced production of bioactive substances required lower inductor concentrations in the culture medium. In the state of the art we found no description of the replacement of the GAL1 gene with the GAL4 gene, which is the object of this invention. To investigate and determine, whether the described replacement influences the heterologous gene expression, we followed the levels of green-fluorescent protein (GFP), and hepatitis B antigen (HBsAg) in the mutant strain, which is another object of this invention, in the gal1 strain, and in the isogenic strain.

GFP has been successfully expressed in several heterologous organisms (Yang et al., 1996, Nature Biotechnology 14, 1246-1251). The gene for GFP was isolated from the jellyfish Aequorea victoria. The protein comprises 238 amino acids, and has a molecular mass of 26.9 kDa (Prasher et al., 1992, Gene 111, 229-233). The protein structure and the fluorophore protection in its interior, are responsible for the protein stability, and its resistance against various physical and chemical agents. The principal advantage of GFP, in comparison with other reporter molecules, is the presence of the fluorophore inside the primary protein structure. Fluorescence is species-nonspecific, and stable. No cofactors or exogenous substrates are required. Fluorescence is easily detectable by UV light, and monitored non-invasively in live cells (Kain et al., 1995, Biotechniques 19, 650-655). GFP exhibits an absorbance peak at 395 nm, a lower peak at 475 nm, and an emission (fluorescence) peak at 509 nm.

GFP is widely used in numerous fields of biotechnology. The easily performed control of the complete bioprocess: starting with clone selection, the control of production conditions, and protein isolation, enables the optimization of all working steps in the production of recombinant protein (Albano et al., 1998, Biotechnology Progress 14, 351-354; Misteli and Spector, 1997, Nature Biotechnology 15, 961-964; Poppenborg et al., 1997, Journal of Biotechnology 58, 79-88).

For all the numerous new expression systems, the yeast S. cerevisiae remains an important host for obtaining various heterologous proteins utilized in the preparation of pharmaceuticals, diagnostics, and vaccines.

An obvious drawback of solutions, described in the state of the art, is that known methods of producing bioactive substances by means of galactose-regulated strains require a higher level of galactose in the nutrient medium, and provide a lower yield of the recombinant product.

The goal of this invention is, accordingly, the construction of a highly productive recombinant strain, which enables a high production of the desired recombinant protein, at low galactose levels in the culture. The recombinant strain, which is the object of this invention, makes feasible a significant increase of the yield of the complete bioprocess, and consequently, lower costs.

In conformance with this invention, this goal is achieved by the replacement of the gene encoding the enzyme for the transformation of the transcription inductor-galactose, into an inactive product, with a gene encoding the transcription factor for the galactose-inducible promoter, as claimed in the independent claims.

### DESCRIPTION OF THE FIGURES

FIG. 1—GFP production from the YCpGAL1-GFP vector (1 ml culture, cell density 2×10⁷/ml) at described culture conditions, in the mutant strain, in gal1, and in the isogenic strain.

FIG. 2—GFP production from the YEp181GAL1-GFP vector (1 ml culture, cell density 2×10⁷/ml) at described culture conditions, in the mutant strain, in gal1, and in the isogenic strain.

### DEFINITIONS

The term “galactose-regulation” refers to the transcription regulation of certain genes by means of effector molecules, or transcription inductors, in the present case by means of galactose.

The term “chromosome” refers to filamentous chromatin structures in the nucleus of eukaryotic cells. They act as carriers of genetic information—inscribed in linear arranged genes.

The term “transcription inductor” refers to an organic molecule inducing the transcriptional activation of structural genes in the cell.

The term “transcription factor” refers to a gene, and to a gene product respectively, regulating the extent and rate of transcription for other, distant genes. In the present case, it is Gal4 regulating the gene transcription of GAL regulon.

The term “promoter” refers to a specific sequence of the DNA molecule—DNA binding site for RNA polymerase II, and other proteins, and molecules required for the transcription initiation.

The term “galactose-inducible promoter” refers to the specific sequence of the DNA molecule—DNA binding site for RNA polymerase II, and other proteins, and inductor galactose required for the transcription initiation.

The term “homologous expression” refers to the expression of a particular organism's own genetic substance or gene inscription. The product is identical to this organism with respect to the structure and activity.

The term “heterologous expression” refers to the expression of a genetic substance or gene inscription, derived from different species, in a specific host organism.

The term “host plasmid” refers to a nonchromosomal genetic element comprising a functional replication site, a genetic marker enabling its recognition, and a structural gene under the control of a strong promoter, enabling the expression of the target protein in a specific host organism.

### DESCRIPTION OF THE INVENTION

This invention is directed to the replacement of the GAL1 gene with the GAL4 gene on the chromosome in the yeast S. cerevisiae. The locus of the GAL1 gene encoding the enzyme galactokinase is, by means of homologous recombination, replaced with the gene for the Gal4 transcription activator. Surprisingly, this enhances the synthesis of recombinant proteins expressed under the control of the GAL1 promoter.

The above replacement relates to strains endowed with galactose-regulation, in which on the chromosome the gene encoding an enzyme that inactivates inductor galactose, has been replaced with a transcription activator of genes driven by galactose-inducible promoter. These strains are preferentially of the families Saccharomycetaceae or Cryptococcaceae, preferentially of the genus Saccharomyces, preferentially the Saccharomyces cerevisiae species.

The above strains are strains, in which the gene encoding an enzyme for the transformation of the transcription inductor into an inactive product is the GAL1 gene.

The above strains are strains, in which the gene encoding the transcription factor is the GAL4 gene. Typically, the synthesis of the GAL4 transcription factor is under the control of the galactose-inducible promoter, preferably the GAL1, GAL2, GAL7, GAL10 or MEL1, preferably GAL1 promoter.

The above strains are strains, characterized in that the galactose-inducible promoter drives the expression of proteins in the presence of a transcription inductor. Preferably, these are GAL1, GAL2, GAL7, GAL10 or MEL1 gene promoters.

The above strains are strains, characterized in that they are hosts for plasmids, used in heterologous or homologous gene expression, and/or are used in the synthesis of bioactive substances or other products of biotechnology. Typically, plasmids used for the heterologous or homologous gene expression, are preferably under the control of the galactose-inducible promoter, preferably the GAL1, GAL2, GAL7, GAL10 or MEL1 gene promoters.

According to this invention, the above strains are used in the synthesis of bioactive substances and other products of biotechnology.

Within the scope of this invention is the use of the above strains for the production of bioactive substances and other products of biotechnology. On the chromosome of these strains, the gene encoding the enzyme for the transformation of the transcription inductor into an inactive product is replaced by a gene encoding the transcription factor for the galactose-inducible promoter, as described above.

The use of the above strains for the production of bioactive substances and other products of biotechnology, with the aid of the galactose-regulated strains, preferably of the family Saccharomycetaceae or Cryptococcaceae, preferably of the genus Saccharomyces, preferably Saccharomyces cerevisiae.

The use of the above strains for the production of bioactive substances and other products of biotechnology with the aid of the strains, characterized in that the gene encoding the enzyme for the transformation of the transcription inductor into an inactive product, is the GAL1 gene replaced by the GAL4 gene encoding the transcription factor.

The use of the above strains for the production of bioactive substances and other products of biotechnology with the aid of the strains, wherein the synthesis of the GAL4 transcription factor is under the control of the galactose-inducible promoter, preferably under the control of the GAL1 promoter.

The use of the above strains for the production of bioactive substances and other products of biotechnology with the aid of the strains, wherein the galactose-inducible promoter drives the expression of proteins in the presence of a transcription inductor, which is preferably chosen from GAL1, GAL2, GAL7, GAL10 or MEL1 gene promoters.

The use of the above strains for the production of bioactive substances and other products of biotechnology with the aid of the strains, which are hosts for plasmids, used in heterologous or homologous gene expression. Preferably, the plasmids are under the control of a galactose-inducible promoter, typically chosen from GAL1, GAL2, GAL7, GAL10 or MEL1 gene promoters.

The products of this invention obtainable with the use of highly-productive recombinant yeast strains, with modified galactose-regulated transcription; are preferably polypeptides, typically homologous and/or heterologous proteins.

The following description of materials and methods is only informative, and serves to illustrate the present invention. Other suitable materials and methods, obvious to those skilled in the art, may be utilized in manufacture.

The following examples represent and illustrate the present invention without, however, limiting the same in any way.

### EXAMPLE 1

### Construction of Highly Productive Recombinant Yeast Saccharomyces cerevisiae Strain with Chromosomal GAL1 by the GAL4 Gene Replacement

The present invention relates to the replacement of GAL genes on chromosome II in yeast S. cerevisiae, using cloning-free process, referred to as ‘delitto perfetto’ (Storici and co., 2001, Nature Biotechnology 19, 773-776). For the GAL gene replacement, other approach can also be used.

The GAL1 inactivation and integration of GAL4 were done as follows. Two long oligonucleotide primers were designed,

GAL1-core-S

SEQ ID NO: 1

(GTTAATATACCTCTATACTTTAACGTCAAGGAGAAAAAACTATAATGga
gctcgttttcgacactgg)
and
GAL1-core-AS

SEQ ID NO: 2

(GAAAAAAATGAGAAGTTGTTCTGAACAAAGTAAAAAAAAGAAGTATACt
ccttaccattaagttgatc).

A counterselectable reporter (CORE) cassette with KlURA3 (counterselectable) and kanMX4 (reporter) was amplified as a 3.2 kb DNA fragment from pCORE (gift from F. Storici) using these 67- and 68-mers, comprising 20 bases homologous to the kanMX4 and KlURA markers, respectively, and 47 and 48 bases complementary to the regions upstream and downstream, respectively, of the yeast GAL1 open reading frame (ORF). The CORE cassette was purified using QIAquick PCR Purification Kit and then transformed by standard recombinant techniques (Wach and co., 1994, Yeast 10, 1793-1804) into the commonly used BY4741 (MATa; his3Δ1; leu2Δ0; met15Δ0; ura3Δ0) yeast strain. Transformants were selected on YEPD (1% Bacto-pepton (Difco), 2% yeast extract, 2% glucose) plates containing 200 mg l⁻¹ of geneticin (G418). Clones with the correct CORE cassette integration were identified by colony PCR, using primers, designed for annealing sequences upstream and downstream from the integration locus and within the marker (CORE) cassette.
The following primers were used:

| | | |
|---|---|---|
| GAL1-A1 | (GGCCCCACAAACCTTCAA), | SEQ ID NO: 3 |
| GAL4-as | (ATGTCAAGGTCTTCTCGAGG) | SEQ ID NO: 4 |
| and | | |
| K3 | (AATATTGTTGATGCGCTGG). | SEQ ID NO: 5 |

GAL4 gene was targeted into yeast strain with correct CORE cassette integration. To prevent non-specific annealing reactions, firstly a GAL4-long gene, containing 145 bp upstream and 533 bp downstream sequence of GAL4 ORF was obtained. The GAL4-long gene (3.3 kbp) was amplified by PCR from yeast genomic DNA (from strain S288C: MAT-α; SUC2; mal; mel; gal2; CUP1; flo1; flo8-1). GAL4 gene used for integration into the target locus was amplified from GAL4-long gene. Cells with correct CORE cassette integration were transformed with this PCR product and 5-fluoroorotic acid resistant (5FOA^{R}), G418 sensitive (G418^{S}) mutants were obtained. Clones with correct GAL4 gene integration were identified by colony PCR, using primers for annealing a sequence upstream from the integration site, a sequence within the kanMX4 marker and a sequence within the GAL4 gene. The following primer oligonucleotides were used:

| | | |
|---|---|---|
| GAL1-A1 | (GGCCCCACAAACCTTCAA), | SEQ ID NO: 3 |
| K3 | (AATATTGTTGATGCGCTGG) | SEQ ID NO: 5 |
| and | | |
| GAL4-as | (ATGTCAAGGTCTTCTCGAGG). | SEQ ID NO: 4 |

Sequence analysis provided additional confirmation that intact GAL4 structural gene has been integrated accurately at the GAL1 locus in the GAL recombinant strain. In first step, GAL1 gene was successfully replaced by CORE cassette, which was, in second step, completely removed by GAL4 open reading frame.

The present invention relates to the highly-productive recombinant strains from Saccharomycetaceae or Cryptococcaceae family, in which gene, encoding an enzyme that inactivates inductor galactose, has been replaced with a transcription activator of genes driven by galactose-inducible promoter. These isogenic strains are preferentially from Saccharomyces genus, preferentially from Saccharomyces cerevisiae species. Gene, encoding an enzyme relates to GAL1, gene encoding transcription activator relates to GAL4 gene. Galactose-inducible promoter relates to GAL1, GAL2, GAL7, GAL10 or MEL1 gene promoter.

The present invention provides the recombinant strains that are host organisms for GAL promoter-driven expression of heterologous genes.

The present invention relates to the production of biological active compounds and other biotechnological products using presented strains that are preferentially from Saccharomyces genus, preferentially from Saccharomyces cerevisiae species. In described production, gene, encoding an enzyme relates to GAL1, gene encoding transcription activator relates to GAL4 gene. Galactose-inducible promoter relates to GAL1, GAL2, GAL7, GAL10 or MEL1 gene promoter.

To evaluate whether GAL gene replacement, relating to the invention, improve protein production, the level of green fluorescent protein (GFP) and Hepatitis B antigen (HbsAg) were analyzed in the GAL recombinant strain, relating to the invention, in gal1 and in non-recombinant strains.

### EXAMPLE 2

### Expression of GFP

YCpGAL1-GFP and YEpGAL1-GFP were constructed as follows. To obtain YCpGAL1 and YEpGAL1, GAL1 promoter and ADH1 terminator were inserted into YCp111 and YEp 181, respectively. Both shuttle vectors contain LEU2 selection marker. YCp111 plasmid contains yeast autonomous replication (ARS1) and centromeric (CEN4) sequences. ARS/CEN vectors are mitotically highly stable, but the copy number is reduced to 1 or 2 per cell. GFP gene was excised from pGFP (Clontech Laboratories) with BamHI, blunt-ended by Mung Bean Nuclease, digested with EcoRI and cloned into the appropriately treated YCpGAL1 vector. YEp181 is based on 2μ that enabling both mitotic stability and high vector copy number. YEpGAL1-GFP was constructed by cloning GFP into BamHI/SmaI restricted YEpGAL1.

The resulting plasmids YCpGAL1-GFP and YEpGAL1-GFP, containing reporter gene, were transformed to the mutant strain, which relates to the invention, gal1 and wild-type, respectively. Transformants were selected on minimal medium without leucine. The yeast cells were grown on YEPD medium, to mid-log phase and then transferred to medium containing 2% galactose. The cells were induced for 18 hours at 30° C., 180 rpm in a reciprocal shaker. For the estimation of GFP in viable cell, yeast were resuspended in TE buffer (10 mM Tris-HCl pH 8.0, 10 mM EDTA) to cell density 2×10⁷/mL. GFP florescence intensity was measured using a fluorescence spectrophotometer at an excitation wavelength of 395 nm and an emission wavelength of 509 nm, respectively (5-nm slit widths). A standard dilution series of GFP provided by Clontech was made in TE buffer and standard curves with a high degree of confidence were established. Standards and samples were measured in triplicate and averaged. All samples were appropriately diluted to give a reading within the range of the standard curve. The level of protein synthesis was determined in the recombinant strain, relating to the invention, in gal1 and in non-recombinant strain.

### 1. Expression of GFP in Yeast Bearing YCpGAL1-GFP

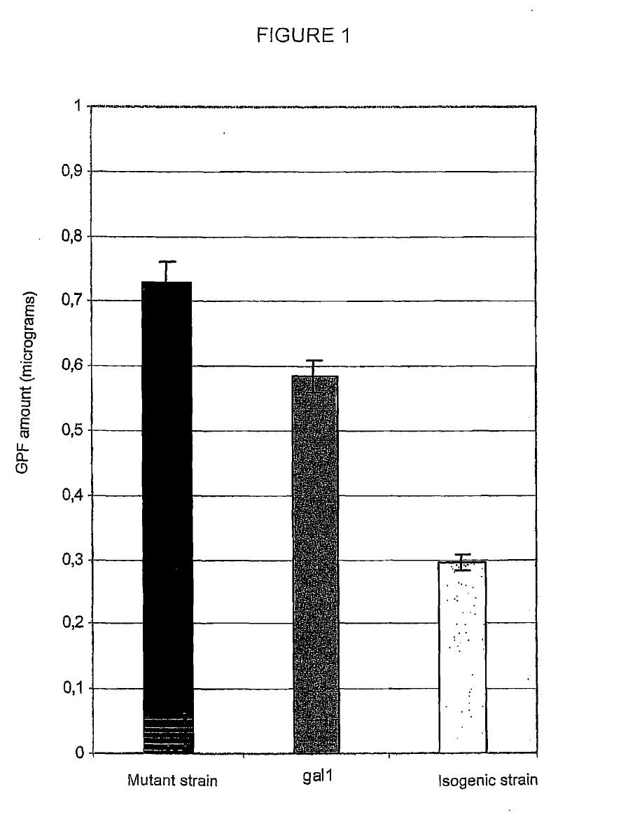

The level of recombinant protein was determined in recombinant strain relating to the invention, in gal1 and in non-recombinant strains. The yeast were cultured and induced as described above. The level of GFP was quantified from established standard curves. Experiments were performed in triplicates. In yeast containing the low-copy plasmid there was an approximately 96% increase in GFP production in the gal1 mutant strain. Moreover, in the resulting recombinant cells that relate to the invention a 145% increase in fluorescence relative to the wild-type was observed. The GFP production in 2×10⁷ cells were 0.297 μg in wild-type, 0.585 μg in gal1 mutant, and 0.729 μg in mutant strain (FIG. 1).

### 2. Expression of GFP in Yeast Bearing YEpGAL1-GFP

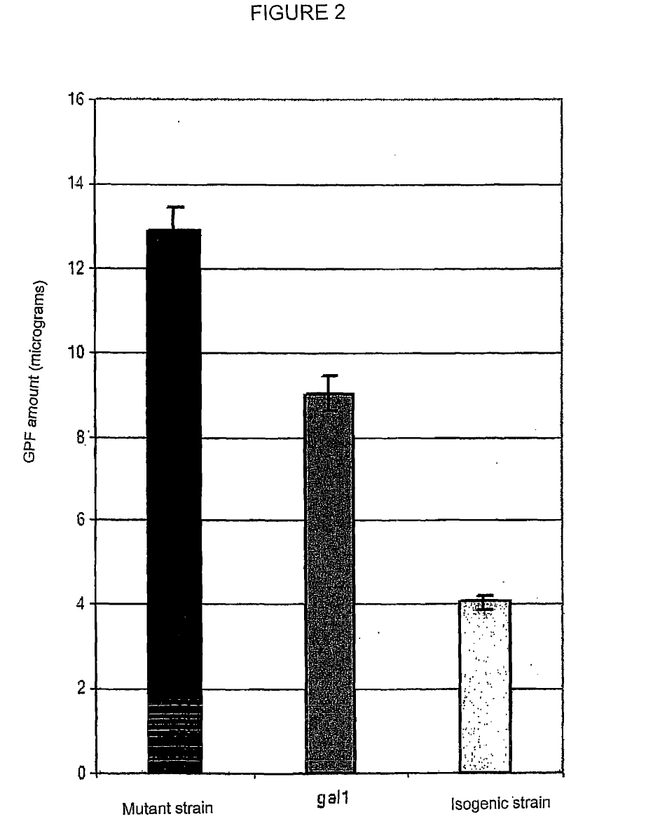

The level of GFP in yeast strains bearing multi-copy plasmid was quantified. The yeast were cultured and induced as described above. Data are the mean from three separate experiments. In yeast containing the multi-copy plasmid there was an approximately 123% increase in GFP production in the gal1 mutant strain. Moreover, in the resulting recombinant cells that relate to the invention a 218% increase in fluorescence relative to the wild-type was observed. The GFP production in 2×10⁷ cells were 4.05 μg in wild-type, 9.03 μg in gal1 mutant, and 12.89 μg in mutant strain (FIG. 2).

### EXAMPLE 3

### Expression of HBsAg

Multi-copy plasmid, bearing gene for Hepatitis B Antigen (HbsAg), were constructed as follows. GAL1 promoter and ADH1 terminator were inserted into YEp181 shuttle vector. Vector was digested with BamHI, blunt-ended by Klenow fragment and subsequently digested with KpnI. HBsAg gene was amplified from pRC/CMV-HBs(S) (gift from Aldevron) by PCR and cloned into expression vector. The resulting plasmid, containing gene for HBsAg, were transformed to the mutant strain, which relates to the invention, gal1 and wild-type, respectively. Transformants were selected on minimal medium without leucine. The yeast cells were grown on YEPD medium, to mid-log phase and then transferred to medium containing 2% galactose. The cells were induced for 18 hours at 30° C., 180 rpm in a reciprocal shaker. Aliquots of cells corresponding to 50 absorbance unit (OD) were collected, washed twice with ice-cold lysis buffer (10 mM PBS pH 7.2, 5 mM EDTA, 0.5 M NaCl, 0.1% (v/v) Triton X-100, 1 mM PMSF) and lysed with glass beads (0.45 μm). Protein concentration was determined by Bradford method with bovine serum albumin as a standard. To estimate the total amount of HbsAg, quantitative dot-blot assay were performed. We used mouse antibody raised against Hepatitis B Surface Antigen (provided by Aldevron). Comparing to commercial available HbsAg standard, total quantity of recombinant protein in yeast transformants was determined. Upon induction by galactose, there was an approximately 2-fold increase in HbsAg production in gal1 mutant strain. Moreover, in the resulting recombinant strain that relates to the invention a 3-fold increase in protein production was observed.

1. A recombinant yeast cell with modified galactose-regulated transcription, wherein a chromosomal gene encoding an enzyme for transformation of a transcription inductor into an inactive product is replaced by a gene encoding a transcription factor for a galactose-inducible promoter.

2. A yeast cell according to claim 1, wherein the yeast cell comprises any one of:
(a) one from the family Saccharomycetaceae or one from the family Cryptococcaceae;
(b) one from the genus Saccharomyces; or
(c) one from the species Saccharomyces cerevisiae.

3-4. (canceled)

5. A yeast cell according to claim 1, wherein the gene encoding the enzyme for the conversion of the transcription inductor into an inactive product comprises the GAL1 gene.

6. A yeast cell according to claim 1, wherein the gene encoding the transcription factor comprises the GAL4 gene.

7. A yeast cell according to claim 6, wherein synthesis of the GAL4 transcription factor is controlled by the galactose-inducible promoter.

8. A yeast cell according to claim 6, wherein synthesis of the GAL4 transcription factor is controlled by any one of a GAL1 promoter, GAL2 promoter, GAL7 promoter, GAL10 promoter, or MEL1 promoter.

9. A yeast cell according to claim 6, wherein a synthesis of the GAL4 transcription factor is controlled by a GAL1 promoter.

10. A yeast cell according to claim 12, wherein the galactose-inducible promoter drives the expression of one or more proteins in the presence of a transcription inductor.

11. A yeast cell according to claim 10, wherein the galactose-inducible promoter comprises any one of a GAL1 gene promoter, GAL2 gene promoter, GAL7 gene promoter, GAL10 gene promoter, or MEL1 gene promoter.

12. A yeast cell according to claim 1, further comprising a plasmid, wherein the plasmid comprises the galactose-inducible promoter operably linked to a heterologous or homologous gene.

13. (canceled)

14. A yeast cell according to claim 12, wherein the galactose-inducible promoter is chosen from the group consisting of GAL1, GAL2, GAL7, GAL10 and MEL1 gene promoters.

15. (canceled)

16. A method for the production of a polypeptide, the method comprising:
(a) providing a recombinant yeast cell with modified galactose-regulated transcription, wherein a chromosomal gene encoding an enzyme for transformation of a transcription inductor into an inactive product is replaced by a gene encoding a transcription factor for a galactose-inducible promoter;
(b) introducing into the cell a nucleic acid, wherein the nucleic acid comprises a sequence encoding the polypeptide operably linked to a galactose-inducible promoter; and
(c) culturing the cell of (b) under conditions permitting expression of the polypeptide.

17. A method according to claim 16, wherein the cells are cultured in the presence of galactose.

18. A method according to claim 16 wherein the recombinant yeast cell comprises any one of:
(a) one from the family Saccharomycetaceae or one from the family Cryptococcaceae;
(b) one from the genus Saccharomyces; or
(c) one from the species Saccharomyces cerevisiae.

19-20. (canceled)

21. A method according to claim 16, wherein the gene encoding the enzyme for the transformation of the transcription inductor into an inactive product comprises the GAL1 gene.

22. A method according to claim 16, wherein the gene encoding the transcription factor comprises the GAL4 gene.

23. A method according to claim 22, wherein synthesis of the GAL4 transcription factor is under the control of the galactose-inducible promoter.

24. A method according to claim 22, wherein synthesis of the GAL4 transcription factor is under the control of the GAL1 promoter.

25. A method according to claim 16, wherein the galactose-inducible promoter drives the expression of one or more proteins in the presence of a transcription inductor.

26. A method according to claim 16, wherein the galactose-inducible promoter comprises any one of a GAL1 gene promoter, GAL2 gene promoter, GAL7 gene promoter, GAL10 gene promoter, or MEL1 gene promoter.

27. A method according to claim 16, wherein the nucleic acid is a plasmid used in heterologous or homologous gene expression.

28. (canceled)

29. A method according to claim 16, wherein the galactose-inducible promoter comprises any one of a GAL1 gene promoter, GAL2 gene promoter, GAL7 gene promoter, GAL10 gene promoter, or MEL1 gene promoter.

30-32. (canceled)

33. A yeast cell according to claim 12, further comprising a polypeptide expressed under the control of the galactose-inducible promoter, wherein the polypeptide is the product of the heterologous or homologous gene.

34. A yeast cell according to claim 33, wherein the polypeptide is a bioactive substance or a biotechnology product.

35. The method according to claim 16, wherein the polypeptide is a bioactive substance or a biotechnology product.
